(19) United States
(12) Patent Application Publication
(10) Pub. No.: US 2024/0324602 A1
(43) Pub. Date: Oct. 3, 2024

(54) METHOD FOR RNAI FUNGICIDE GENERATING AND THE RNAI FUNGICIDE THEREOF

(71) Applicant: XIAOMO BIOTECH LIMITED, HONG KONG (CN)

(72) Inventors: LINFENG HUANG, HONG KONG (CN); PANPAN HUANG, HONG KONG (CN); YUTIAN REN, HONG KONG (CN); SHANQIN LI, HONG KONG (CN)

(21) Appl. No.: 18/740,972

(22) Filed: Jun. 12, 2024

Related U.S. Application Data
(63) Continuation of application No. PCT/CN2022/116331, filed on Aug. 31, 2022.

(51) Int. Cl. A01N 63/60; A01P 3/00; C12N 15/113

(52) U.S. Cl. CPC A01N 63/60; A01P 3/00; C12N 15/113; C12N 2310/14; C12N 2320/12; C12N 2330/31

(57) ABSTRACT

A method for RNAi fungicide generating and the RNAi fungicide thereof. The method includes culturing bacteria in multi-aperture culture plate to express siRNA-binding polypeptide; extracting the siRNA-binding polypeptide from the bacteria; generating a pro-siRNA library product according to the siRNA-binding polypeptide, screening at least one target gene from the pro-siRNA library product to generate RNAi fungicide.

### TECHNICAL FIELD

The technology described herein relates to methods of generating customized RNAi fungicide.

### BACKGROUND

RNA interference (RNAi) by double-stranded (ds) small interfering RNAs (siRNA) suppresses gene expression by inducing the degradation of mRNAs bearing complementary sequences. Transfection of synthetic siRNAs into eukaryotic cells to silence genes has become an indispensable tool to investigate gene function, and siRNA-based therapy is being developed to knockdown genes implicated in disease.

A novel siRNA design and production system has been invented by Dr. Linfeng Huang (Huang et al, Nat Biotechnology, 2013, 31 (4): 350-6; Huang & Lieberman, Nat Protocol, 2013, 8 (12): 2325-36). This system utilizes the unique function of the protein p19, which can bind to and stabilize 21-nt double stranded RNA species produced by endogenous RNase III in Escherichia coli (E. coli), producing a pool of siRNAs within a certain selected gene sequence. Those siRNAs produced in E. coli are called pro-siRNA for prokaryotic siRNA.

The pathogen Botrytis cinerea causes gray mold in more than 200 crop species (mainly dicots) and annual losses of $10 billion to $100 billion worldwide. Agrochemicals, as the most common way to control those pathogen fungi, have poor specificity and high toxicity for the environment and human health. Despite large increases in pesticide use, crop losses have not significantly decreased during the last 40 years.

Therefore, there is a great need for effective and safe control measures for crop protection.

### SUMMARY

The technology described herein is directed to RNAi fungicide and the generating method thereof relating to the crop protection.

In an aspect, a RNAi fungicide includes: a siRNA-binding polypeptide; wherein the nucleic acid sequence of the siRNA substantially complementary to a target RNA.

In an embodiment, the polypeptide includes protein 19 and a purification tag.

In an embodiment, the purification tag is a histidine tag.

In an aspect, the method for RNAi fungicide generating includes culturing bacteria in multi-aperture culture plate to express siRNA-binding polypeptide, extracting the siRNA-binding polypeptide from the bacteria, generating a pro-siRNA library product according to the siRNA-binding polypeptide to generate RNAi fungicide.

In an embodiment, the method further includes culturing fungus, constructing a plasmid for expressing pro-siRNA and transforming the plasmid to bacteria.

In an embodiment, the fungus is selected from Botrytis cinerea.

In an embodiment, the step of constructing a plasmid further includes isolating mRNA of the fungus, acquiring cDNA fragments from mRNA conversion and connecting the cDNA fragments into the plasmid.

In an embodiment, the multi-aperture culture plates are 96-well plates.

In an embodiment, the bacteria are Escherichia coli cell.

In an embodiment, the step of extracting the siRNA-binding polypeptide from the bacteria further includes purifying the siRNA-binding polypeptide by a histidine tag.

One advance of the RNAi fungicide disclosed by the embodiments is that the fungicide is nontoxic to human and biodegradable and the sequence is adaptable to overcome resistance.

### BRIEF DESCRIPTION OF THE DRAWINGS

In order to more clearly illustrate the technical solutions of the embodiments of the present application, a brief description will be given below of the accompanying drawings which are required to be used in the embodiments. It is to be understood that the following drawings illustrate only some embodiments of the present application and are therefore not to be considered as limiting the scope. For ordinary technical personnel in this field, other relevant drawings can also be obtained according to these drawings without involving any inventive effort.

FIG. 1 is a flow chart of a method for pro-siRNA library generating according to embodiments of the present application.

FIG. 2A is schematic diagram of the conversion result of isolated mRNA according to embodiments of the present application.

FIG. 2B is schematic diagram of the pro-siRNA express vector construction according to embodiments of the present application.

FIG. 2C is schematic diagram of the result of transformation of the vector to bacteria according to embodiments of the present application.

FIG. 2D is schematic diagram of the pro-siRNA quality control by random PAGE-GEL visualization according to embodiments of the present application.

FIG. 2E is schematic diagram of representative concentration information of one plate of pro-siRNA Library according to embodiments of the present application.

FIG. 3 is a flow chart of the method for RNAi fungicide generating according to embodiments of the present application.

FIG. 4A is a schematic diagram of primary screen result according to embodiments of the present application.

FIG. 4B is a schematic diagram of validation result according to embodiments of the present application.

FIG. 4C is a schematic diagram of sequence analysis result according to embodiments of the present application.

FIG. 4D is a schematic diagram of the pro-siRNA examination on PAGE-Gel according to embodiments of the present application.

FIG. 4E is a schematic diagram of the insert examination of plasmid on Agarose-Gel according to embodiments of the present application.

FIG. 5 is a flow chart of the validation experiment of the RNAi fungicide according to embodiments of the present application.

FIG. 6A is a schematic diagram of the representative bright field images captured by HCS according to embodiments of the present application.

FIG. 6B is a schematic diagram of the germ tubes analysis result output by the HCS according to embodiments of the present application.

FIG. 7A is a schematic diagram of lesion area of different pro-siRNA fungicide on green beans plant according to embodiments of the present application.

FIG. 7B is a schematic diagram of validation result of different pro-siRNA fungicide shown in FIG. 7A.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of this description.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e., a non-detectable level as compared to a reference level. In the context of a marker or symptom is meant a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “proteins” and “polypeptides” are used interchangeably herein to designate a series of amino acid residues connected to the other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. A gene may or may not include regions preceding and following the coding region, e.g., 5′ untranslated (5′UTR) or “leader” sequences and 3′ UTR or “trailer” sequences, as well as intervening sequences (introns) between individual coding segments (exons).

The term “operatively linked” includes having an appropriate start signal (e.g., ATG) in front of the polynucleotide sequence to be expressed and maintaining the correct reading frame to permit expression of the polynucleotide sequence under the control of the expression control sequence, and, optionally, production of the desired polypeptide encoded by the polynucleotide sequence. In some examples, transcription of a nucleic acid modulatory compound is under the control of a promoter sequence (or other transcriptional regulatory sequence) which controls the expression of the nucleic acid in a cell-type in which expression is intended. It will also be understood that the modulatory nucleic acid can be under the control of transcriptional regulatory sequences which are the same or which are different from those sequences which control transcription of the naturally occurring form of a protein.

The term “isolated” or “purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell or secreted in the case of secreted polypeptides. A chemically synthesized nucleic acid or polypeptide or one synthesized using in vitro transcription/translation is considered “isolated.”.

The term “complementary” or “complementary base pair” refers to A: T and G: C in DNA and A: U in RNA. Most DNA consists of sequences of nucleotide only four nitrogenous bases: base or base adenine (A), thymine (T), guanine (G), and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

Other than in the operating examples, or where otherwise indicated, all numbers expressing quantities of ingredients or reaction conditions used herein should be understood as modified in all instances by the term “about.” The term “about” when used in connection with percentages can mean±1%.

The term “includes” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

The term “consisting essentially of” refers to those elements required for a given embodiment. The term permits the presence of elements that do not materially affect the basic and novel or functional characteristic(s) of that embodiment.

The singular terms “a” “an” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.”.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention can be performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Referring to FIG. 1 which is illustrated the flow chart of the method for RNAi fungicide generating, the method includes follow steps.

Firstly, culturing fungus (S110), such as Botrytis cinerea. Then, isolating mRNA of the fungus and converting the mRNA to cDNA fragment (S120).

Secondly, connecting the cDNA fragment into the vector to construct a special expressing vector of target pro-siRNA (S130). Alternatively, the vector may further include other nucleic acid in different function, such as the nucleic acid encoding purification tag or the nucleic acid encoding protein 19.

The term “vector”, as used herein, refers to a nucleic acid construct designed for delivery to a host cell or transfer between different host cells. As used herein, a vector can be viral or non-viral. Many vectors useful for transferring exogenous genes into target cells are available, e.g. the vectors may be episomal, e.g., plasmids, virus derived vectors or may be integrated into the target cell genome, through homologous recombination or random integration. In some embodiments, a vector can be an expression vector. As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms. The nucleic acid incorporated into the vector can be operatively linked to an expression control sequence when the expression control sequence controls and regulates the transcription and translation of that polynucleotide sequence. In some embodiments, the dsRNA and the nucleic acid encoding the siRNA-binding polypeptide can be within the same operon. In some embodiments, the dsRNA and the nucleic acid encoding the siRNA-binding polypeptide can be within separate operons.

As used herein, “protein 19” can be also named as p19 polypeptides. “protein 19” in particular but not limited to the p19 polypeptide such as tombusvirus p19 (NCBI Gene ID: 1493957) are able to bind to the siRNA and in particular include those as disclosed in US 2015/0337306A1 which are incorporated herein by reference. The p19 polypeptide as disclosed in US 2015/0337306A1, i.e. selected from a p19 polypeptide such as tombusvirus p19 polypeptide.

The term “protein 19” refers to a viral protein which binds specifically to dsRNAs and which suppresses RNAi-mediated host plant viral defenses. The sequences of p19 polypeptides from a number of species are known, e.g. tombusvirus p19 (NCBI Gene ID: 1493957). In some embodiments, the p19 polypeptide can be tombusvirus p19. Non-limiting examples of p19 homologues include Carnation Italian ringspot virus P19; Tomato bushy stunt virus p19; Artichoke mottled crinkle virus p19; Lisianthus necrosis virus p19; Pear latent virus p19; Cucumber Bulgarian virus p19; Cucumber necrosis virus p19; Pelargonium necrotic spot virus p19; Cymbidium ringspot virus p19; Lisianthus necrosis virus p19; Lettuce necrotic stunt virus p19; Maize necrotic streak virus p19; Grapevine Algerian necrosis virus p19; and Grapevine Algerian latent virus p19. A p19 polypeptide can comprise mutants, variants, homologues, and functional fragments of wildtype p19 polypeptides.

Preferably, the vector further comprises a “siRNA-binding polypeptide expression cassette” including a promoter and a sequence encoding a siRNA-binding polypeptide and optionally a siRNA-generating enzyme, in particular a ribonuclease like an Escherichia coli (E. coli) RNase III. One of skill in the art will understand that the method of the present invention could also use sequences encoding a siRNA-generating enzyme such as a RNase III from any other bacterial species. In preferred embodiments of the present invention, the vector is a plasmid further comprising a siRNA-binding polypeptide expression cassette including a promoter, a sequence encoding a siRNA-binding polypeptide and a sequence encoding a siRNA-generating enzyme, wherein the siRNA-binding polypeptide is a p19 polypeptide and the siRNA-generating enzyme is an E. coli RNase III.

Thirdly, transforming the vector to bacteria and culturing the bacteria (S140), such that siRNA-binding polypeptide can be expressed. In some embodiments, the nucleic acid sequence of siRNA substantially complementary to the target RNA (i.e., the mRNA isolated from the Botrytis cinerea).

Alternatively, the target RNA may include, but not limited to, several RNA as follow:
hypothetical protein (BCIN_01g06470);
hypothetical protein (BCIN_05g00180);
Bcaco1 (BCIN_02g05920);
hypothetical protein (BCIN_06g04170);
Bcbac (BCIN_15g02590);
Bcbmp1 (BCIN_02g08170);
Bcplc1 (BCIN_05g02840);
Bcbcg1 (BCIN_05g06770).

Alternatively, the bacteria can be cultured in 96-well plate.

As used herein, “substantially complementary” refers to a first nucleotide sequence having at least 90% complementarity over the entire length of the sequence with a second nucleotide sequence, e.g. 90% complementary, 95% complementary, 98% complementary, 99% complementary, or 100% complementary. Two nucleotide sequences can be substantially complementary even if less than 100% of the bases are complementary, e.g. the sequences can be mismatched at certain base.

As used herein, the term “target RNA” or “target gene” refers to a nucleic acid sequence present in a cell (i.e. the “target cell”). The target RNA comprises a target sequence to which one strand of a siRNA according to the methods and compositions described herein binds, thereby causing RNAi silencing of the target RNA. The target cell can be the bacterial cell comprising a siRNA-binding polypeptide or another cell, either prokaryotic or eukaryotic. The target sequence can be an RNA that can be translated (i.e. it can encode a polypeptide, e.g. mRNA) or it can be an RNA that is not translated (i.e. a non-coding RNA). In some embodiments, the target sequence can be any portion of an mRNA. In some embodiments, the target sequence can be a sequence endogenous to the cell. In some embodiments, the target sequence can be a sequence exogenous to the cell. In some embodiments, the target sequence can be sequence from an organism that is pathogenic to the target cell, e.g. the target sequence can be sequence from a viral, bacterial, fungal, and/or parasitic origin. In some embodiments, the target sequence is a viral nucleotide sequence.

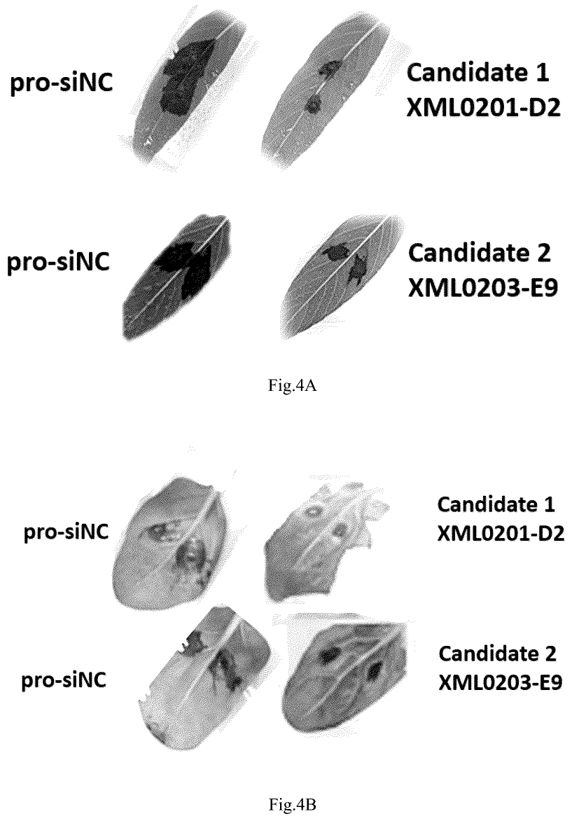

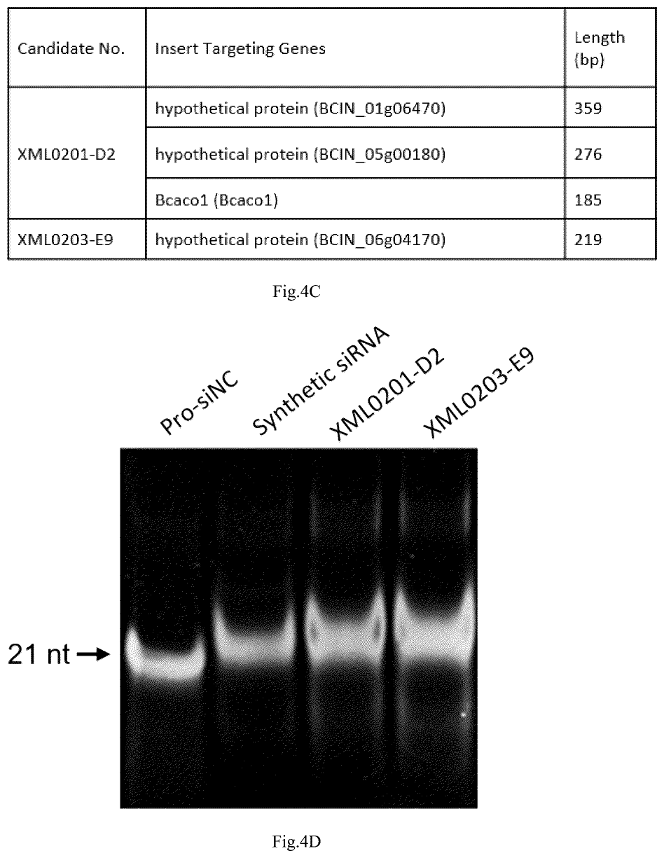

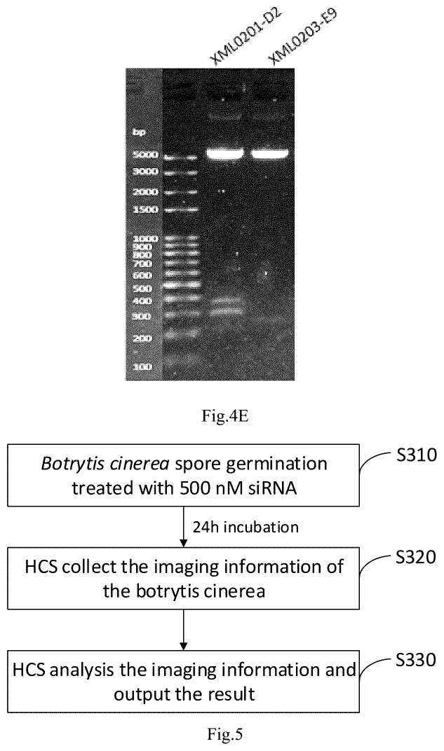

Finally, extracting the siRNA-binding polypeptide from the bacteria (S150) and generating a pro-siRNA library (S160). Accordingly, at least one target gene can be screened from the pro-siRNA library product and the customized RNAi fungicide can be generated. Preferably, the target gene can be the gene sequence shown in the table of FIG. 4C, according to the highlighted gene screening results.

As used herein, the term “extracting” as used herein means separating the siRNA from other components such as from the bacterial cells and other DNA or RNA sequences or polypeptides that are present resulting from the materials used and conditions applied for producing the siRNA. In particular, isolating the siRNA comprises extracting and purifying the siRNA.

In some embodiments, after the step of generating the pro-siRNA library, the method further includes purifying pro-siRNAs from the pro-siRNA library and testing their effect on inhibiting the growth of fungus.

Specifically, the inhibiting effect of the purified pro-siRNAs can be tested by means of high-content screen in plates and antifungal effects on the leaves.

Further preferred, the siRNA-binding polypeptide has a purification tag suitable for extracting the siRNA-binding polypeptide from the bacteria. The purification tag can bind to another moiety such as on a matrix or a resin with affinity for the purification tag such as Ni-NTA resin. Particular purification tags include histidine tags (“His-tagged”) such as disclosed in US 2015/0337306A1 which are incorporated herein by reference. The siRNA-binding polypeptide is in particular a His-tagged p19 polypeptide.

The vector is in particular able to express a siRNA-binding polypeptide, in particular a p19 polypeptide such as His-tagged p19 polypeptide, and a siRNA-generating enzyme like a RNase III or siRNA-binding polypeptide, in particular p19 such as His-tagged p19, fused to a siRNA-generating enzyme like an E. coli RNase III. Such expression of siRNA-generating enzyme like an E. coli RNase III will enhance the siRNA production.

In an embodiment, the siRNAs can be generated from the dsRNA comprising a nucleic acid sequence substantially complementary to a target RNA. As used herein, the term “siRNA” refers to a nucleic acid that forms an RNA molecule comprising two individual strands of RNA which are substantially complementary to each other. Typically, the siRNA is at least about 15-40 nucleotides in length (e.g., each complementary sequence of the double stranded siRNA is about 15-40 nucleotides in length, and the double stranded siRNA is about 15-40 base pairs in length, preferably about 19-25 base nucleotides, e.g., 19, 20, 21, 22, 23, 24, or 25 nucleotides in length). In some embodiments, a siRNA can be blunt-ended. In some embodiments, a siRNA can comprise a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. The siRNA molecules can also comprise a 3′ hydroxyl group. In some embodiments, the siRNA can comprise a 5′ phosphate group. A siRNA has the ability to reduce or inhibit expression of a gene or target RNA when the siRNA is present or expressed in the same cell as the target gene, e.g. the target RNA. siRNA-dependent post-transcriptional silencing of gene expression involves cutting the target RNA molecule at a site guided by the siRNA.

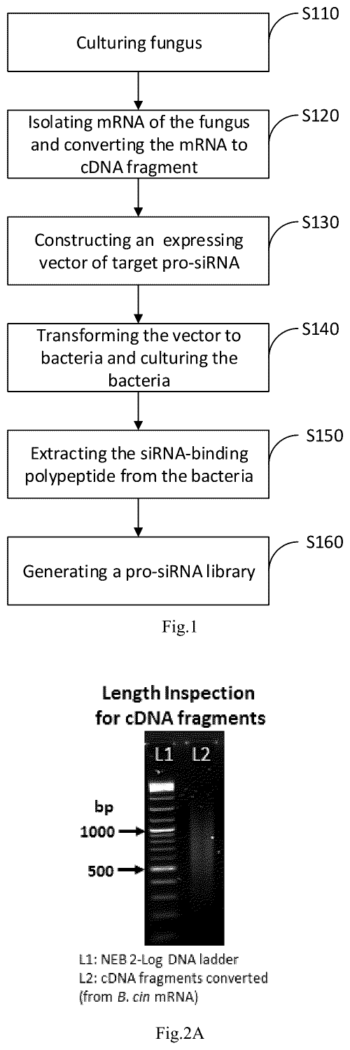

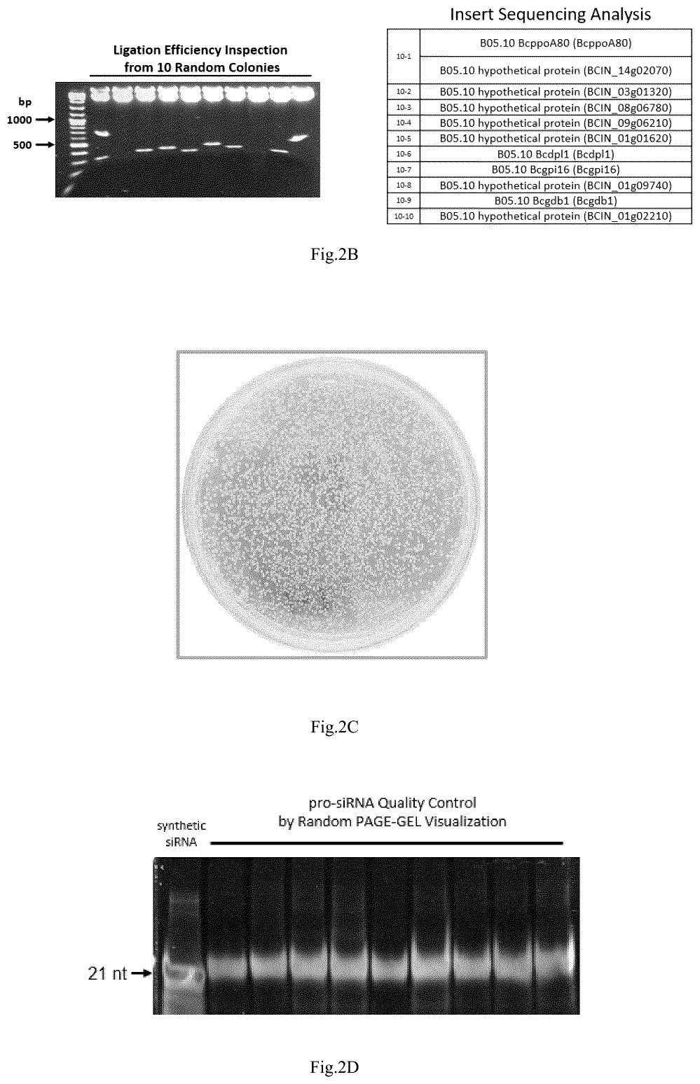

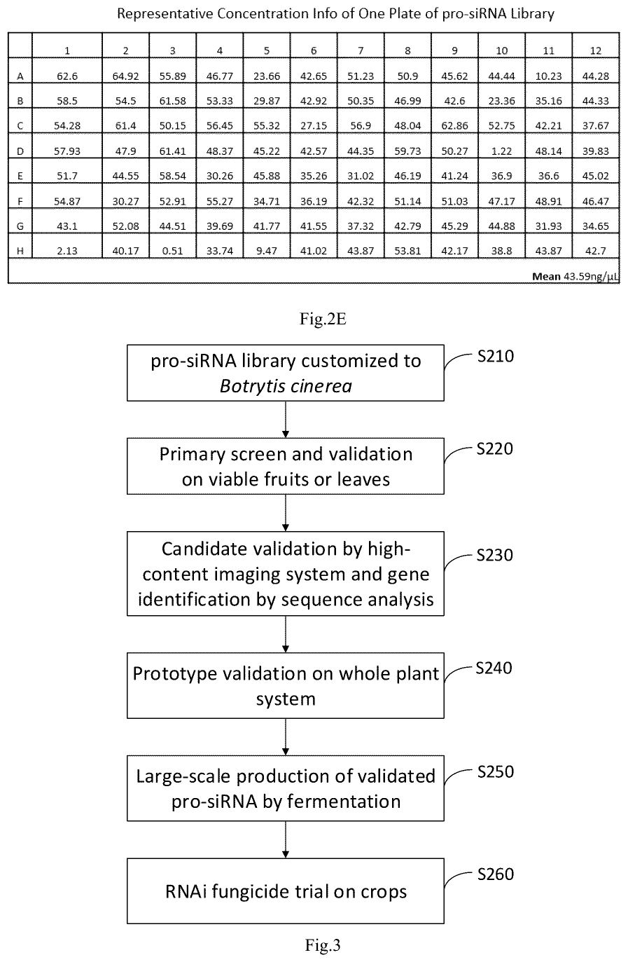

Referring to FIG. 2E, there is the example for pro-siRNA library generation. FIG. 2A illustrates the result of the step of mRNA isolation and cDNA conversion. FIG. 2B illustrates the result of the step of pro-siRNA expressing vector construction. FIG. 2C illustrates the result of the step of transformation of the vector to bacteria. FIG. 2D illustrates the result of pro-siRNA quality control by random PAGE-GEL visualization. FIG. 2E illustrates representative concentration information of one plate of pro-siRNA Library.

It should be noted that the methods of creating bacterial libraries, and/or libraries of compounds isolated from bacterial cells are well known in the art. By way of non-limiting example, a bacterial cell library can be in the form of a plurality of multi-well plates, with each well of a plate comprising a clonal bacterial population. The clonal bacterial populations can be provided in media or in glycerol stocks. In some embodiments, a library can comprise multiple wells which comprise identical clonal populations, i.e. a clonal population can appear multiple times in a library. In some embodiments, a library can comprise a plurality of multi-well plates, with each well of a plate comprising one or more siRNA species (e.g. a siRNA species or a population of siRNA species) isolated from one or more clonal bacterial populations. Methods of isolating nucleic acids from bacterial cells are well known in the art and examples are described elsewhere herein. In some embodiments, libraries can be created using automated and/or high-throughput methods, e.g. robotic colony-picking.

In some embodiments, a library can comprise pooled samples, e.g. multiple clonal bacterial populations, multiple isolated siRNAs, or multiple isolated populations of siRNA species can be pooled so that a smaller number of samples must be initially screened. The individual components of a “positive” pooled can be subsequently screened separately.

In some embodiments, a library can comprise 10 or more pools of, populations of, and/or individual siRNA species (e.g. isolated or present within bacterial cells), e.g. 10 or more, 100 or more, 1,000 or more, 10,000 or more, or 100,000 or more pools of, populations of, and/or individual siRNA species.

In some embodiments, a library can comprise a plurality of populations of siRNAs, wherein each population of siRNAs can silence at least one target RNA of a target set. A target set of RNAs can comprise, e.g. the transcriptome of a cell, the transcriptome of an organism, the transcriptome of a cell and/or organism in a specific state (e.g. a diseased organism or an organism at a specific stage of development) or a subtractive transcriptome (e.g. all the transcripts present in a cell under one condition but which are not present in the cell in a second condition).

FIG. 3 illustrates the flow chart of the method for RNAi fungicide application. Referring to FIG. 3, the method includes follow steps.

Firstly, the pro-siRNA library customized to Botrytis cinerea has been constructed (S210).

Then, primary screen operation and validation have been executed on viable fruits or leaves (S220).

For instance, as show in FIG. 4A, there are candidate 1 (named as XML0201-D2) and candidate 2 (named as XML0203-E9) has been screened according to the primary screen result.

Secondly, the candidate validation has been completed by high-content imaging system (HCS) and the gene identification has been completed by sequence analysis (S230).

FIG. 4B illustrates the validation on spinach leaves of the candidate 1 and candidate 2. The result of the sequence analysis for candidate 1 and candidate 2 has been illustrated in FIG. 4C.

FIG. 4D illustrates the result of the pro-siRNA examination on PAGE-Gel. FIG. 4E illustrates the result of the insert examination of plasmid on Agarose-Gel.

Thirdly, the protype validation has been executed on whole plant system (S240). Since the pro-siRNA validated, large-scale production has been completed by fermentation (S250).

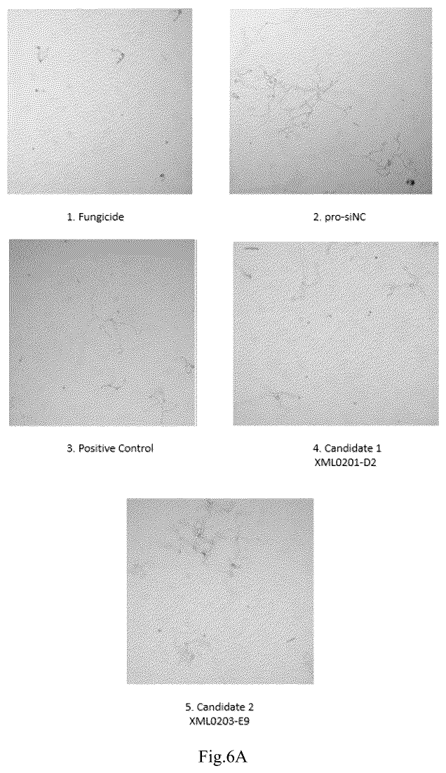

Finally, the production of RNAi fungicide trial on crops (S260). FIG. 5 illustrates the flow chart of validation experiment of the RNAi fungicide.

In some embodiments, referring to FIG. 5, the Botrytis cinerea spore germination can be treated with 500 nm siRNA (S310). After 24 h incubation, HCS collect the imaging information of the Botrytis cinerea (S320). Finally, HCS analysis the imaging information and output the result.

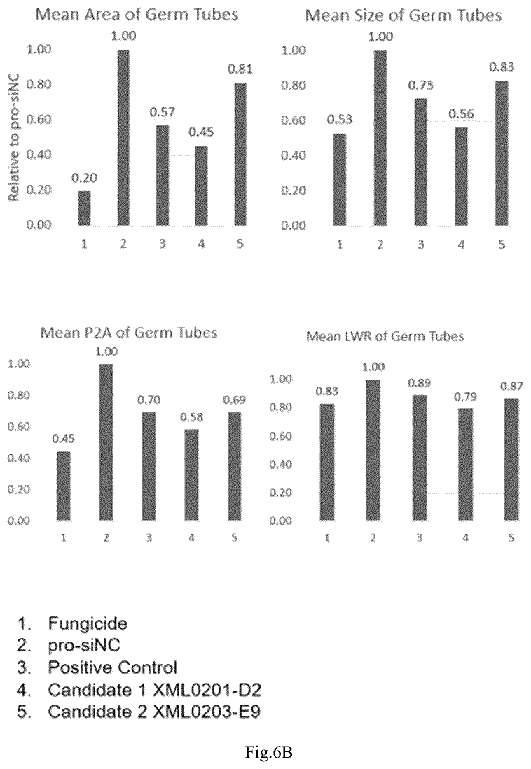

FIG. 6A illustrates the representative bright field images captured by HCS. FIG. 6B illustrates the germ tubes analysis result output by the HCS. As shown in FIGS. 6A and 6B, the candidate 1 and candidate 2 can inhibits the growth of fungi.

In general, a platform to produce personalized library of pro-siRNAs specifically targeting the plant pathogen, gray mold Botrytis cinerea, is successfully established, based on the patented pro-siRNA technologies.

All steps within the RNAi production platform have passed QC examination, which guarantee a qualified condition for downstream applications.

Under the established platform disclosed by the application, a transcriptome-wide loss-of-function screen has been produced and applied to gray mold Botrytis cinerea.

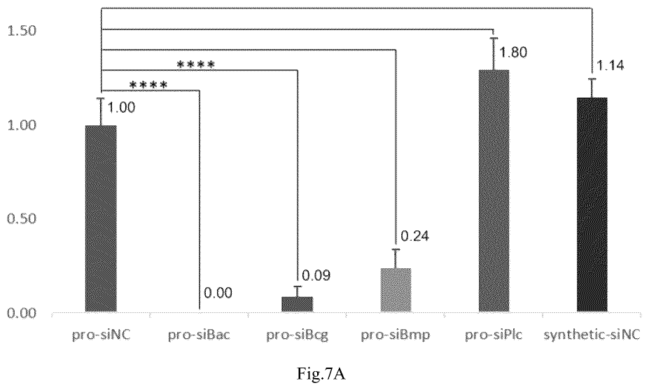

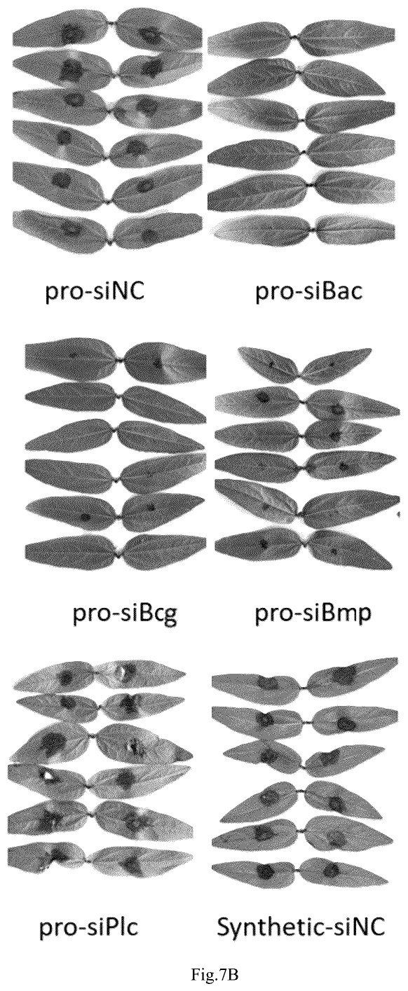

FIGS. 7A and 7B illustrate the lesion area of different pro-siRNA fungicide on green beans plant. The label of pro-siNC and synthetic-siNC are the control group. The label of pro-siBac represents that the target RNA of pro-siRNA fungicide is Bcbac (BCIN_15g02590). The label of pro-siBcg represents that the target RNA of pro-siRNA fungicide is Bcbcg1 (BCIN_05g06770). The label of pro-siBmp represents that the target RNA of pro-siRNA fungicide is Bcbmp1 (BCIN_02g08170). The label of pro-siPlc represents that the target RNA of pro-siRNA fungicide is Bcplc1 (BCIN_05g02840). As shown in FIGS. 7A and 7B, compared with the control group, the pro-siRNA fungicide can effective reduce the lesion area of the green beans plant.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. A RNAi fungicide comprising siRNA attached to or isolated from siRNA-binding polypeptide; wherein the nucleic acid sequence of the siRNA substantially complementary to a target RNA.

2. The RNAi fungicide of claim 1, wherein the polypeptide comprises protein 19 and a purification tag.

3. The RNAi fungicide of claim 2, wherein the purification tag is histidine tag.

4. The RNAi fungicide of claim 1, wherein the target RNA comprises, but not limited to:
hypothetical protein (BCIN_01g06470);
hypothetical protein (BCIN_05g00180);
Bcaco1 (BCIN_02g05920);
hypothetical protein (BCIN_06g04170);
Bcbac (BCIN_15g02590);
Bcbmp1 (BCIN_02g08170);
Bcplc1 (BCIN_05g02840); and
Bcbcg1 (BCIN_05g06770).

5. A method for RNAi fungicide generating, comprising:
culturing bacteria in multi-aperture culture plate to express siRNA-binding polypeptide;
extracting the siRNA-binding polypeptide from the bacteria;
generating a pro-siRNA library product according to the siRNA-binding polypeptide;
screening at least one target gene from the pro-siRNA library product to generate RNAi fungicide.

6. The method of claim 5, wherein the method further comprises:
purifying pro-siRNAs from the pro-siRNA library;
testing the effect of the purified pro-siRNAs on inhibiting the growth of fungus.

7. The method of claim 6, wherein the effect of the purified pro-siRNAs on inhibiting the growth of fungus test by means of high-content screen in plates and antifungal effects on the leaves or other plant tissues.

8. The method of claim 5, wherein the method further comprises:
culturing fungus;
constructing a plasmid for expressing pro-siRNA;
transforming the plasmid to bacteria.

9. The method of claim 8, wherein the step of constructing a plasmid further comprises:
isolating mRNA of the fungus;
acquiring cDNA fragments from mRNA conversion;
connecting the cDNA fragment into the plasmid.

10. The method of claim 6, wherein the multi-aperture culture plate is 96-well plates.

11. The method of claim 10, wherein the bacteria are Escherichia coli cell.
